(19) United States
(12) Patent Application Publication
(10) Pub. No.: US 2010/0269216 A1
(43) Pub. Date: Oct. 21, 2010

(54) NETWORK POPULATION MAPPING

(75) Inventors: Zhigang Guo, Champaign, IL (US); Venkata Krishna Kishore, Bloomington, IL (US); Suresh Babu Kadaru, Northfield, MN (US); Min Li, Champaign, IL (US); Todd Lee Warner, Eagan, MN (US); Homer Gene Caton, Madrid, IA (US)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Participations AG, Basel (CH)

(21) Appl. No.: 12/425,122

(22) Filed: Apr. 16, 2009

(51) Int. Cl. A01H 1/06; C12Q 1/68; A01H 5/00

(52) U.S. Cl. 800/278; 435/6; 800/295

(57) ABSTRACT

Provided herein are methods for mapping quantitative trait loci in a connected population of organisms. The invention includes evaluating associations between markers and a trait of interest using network population mapping (NPM). The methods include assembling a network of individual members for association mapping, wherein the members are connected at the allelic level. Members of the network are grouped according to a shared haplotype at one or more marker loci, and the network can be used to identify or validate QTL within the chromosomal region surrounding or flanked by the marker loci. The methods further include a means for estimating and ranking the effects of multiple alleles across the mapping population. Further provided is a novel simple interval mapping model as well as a novel composite interval mapping model for evaluating allele-specific associations across a connected mapping population.

### FIELD OF THE INVENTION

This invention relates to molecular genetics, particularly to methods for evaluating an association between a genetic marker and a phenotype in a population connected with other populations.

### BACKGROUND OF THE INVENTION

Multiple experimental paradigms have been developed to identify and analyze quantitative trait loci (QTL) (see, e.g., Jansen (1996) Trends Plant Sci 1:89). A quantitative trait locus (QTL) is a region of the genome that codes for one or more proteins and explains a significant proportion of the variability of a given phenotype that may be controlled by multiple genes. The majority of published reports on QTL mapping in crop species have been based on the use of the bi-parental cross. Typically, these paradigms involve crossing one or more parental pairs, which can be, for example, a single pair derived from two inbred strains, or multiple related or unrelated parents of different inbred strains or lines, each of which exhibits different characteristics relative to the phenotypic trait of interest.

To perform QTL detection, the general practice has been to develop a few specific bi-parental mapping populations of large size, in order to guarantee sufficient power of the tests. Typically, this experimental protocol involves deriving 100 to 300 segregating progeny from a single cross of two divergent inbred lines (e.g., selected to maximize phenotypic and molecular marker differences between the lines). The parents and segregating progeny are genotyped for multiple marker loci and evaluated for one to several quantitative traits (e.g., disease resistance). QTL are then identified as significant statistical associations between genotypic values and phenotypic variability among the segregating progeny.

Analyzing these large specific populations individually has clearly been successful in detecting QTL in plants (Kearsey and Farquhar 1998, Heredity 80:137-142; Asins 2002, Plant Breed 121:281-291; Bernardo 2002, Quantitative traits in plants. Stemma, Woodbury) and some QTL could be cloned, in particular not only in rice and tomato (Takahashi et al. 2001, Proc Natl Acad Sci USA 98:7922-7927; Kojima et al. 2002, Plant Cell Physiol 43:1096-1105; Liu et al. 2002, Proc Natl Acad Sci USA 99:13302-13306; Liu et al. 2003, Plant Physiol 132:292-299) but also in maize (Doebley et al. 1997, Nature 386:485-488). However, the QTL identified in these populations may not be broadly applicable to non-related populations. This problem limits the use of bi-parental mapping populations for QTL detection.

### SUMMARY OF THE INVENTION

Provided herein are methods for mapping quantitative trait loci in a connected population of either plant or animal organisms. The invention comprises evaluating or validating associations between markers and a trait of interest using network population mapping (NPM). The methods comprise assembling a network of individual members for association mapping, wherein the members are connected at the allelic level. Members of the network share a common allele at one or more marker loci, and the network can be used to identify or validate QTL within the chromosomal region surrounding or flanked by the marker loci. The methods further comprise a means for estimating and ranking the effects of multiple alleles across the mapping population.

The methods further comprise a novel simple interval mapping model as well as a novel composite interval mapping model for evaluating allele-specific associations across a connected mapping population.

QTL markers identified, selected, or validated using the methods of the invention can be used in marker assisted breeding and selection, as genetic markers for constructing genetic linkage maps, to isolate genomic DNA sequence surrounding a gene-coding or non-coding DNA sequence, to identify genes contributing to a trait of interest, and for generating transgenic organisms having a desired trait. All favorable alleles existing in the mapping population can be utilized for marker assisted breeding to improve the efficiency of the process.

### BRIEF DESCRIPTION OF THE FIGURES

The following figures are exemplary, and are not intended to describe the full scope of the invention.

FIG. 1 is an exemplary diagram of how the allelic connection structure is considered in the model for network population mapping (NPM) in contrast to general connected population mapping (CPM). In CPM, each parent (P) is assumed to hold a different allele. In NPM, common alleles are defined by a haplotype at a specific locus. Thus, in this example, the effects of four different alleles are needed to estimate in CPM (assuming one allele per parent in a 4-parent cross), while only two allelic effects are dealt with in NPM (i.e., the actual number of different alleles observed at that locus). MP=mapping population.

FIG. 2A depicts an example of using haplotypes of two flanking markers to infer alleles of a QTL. The left side represents the haplotypes defined by two adjacent marker loci of three parents. In this example, each haplotype is assumed to represent a different QTL allele within the interval flanked by the two markers. Therefore, in total there are three QTL alleles in this example (a, b, and c). The right side shows the possible segregation of marker and QTL alleles in double haploid (DH) populations derived from the three common parents P₁, P₂ and P₃. These combined allele calls will be used for the NPM analysis. The power for QTL detection in NPM comes from combining shared alleles in the DH lines used in the example.

FIG. 2B depicts an example of inferring QTL probability conditional on two flanking markers in each bi-parental population using a consensus map. The top of the figure shows the genotypic segregation of QTL alleles within the interval defined by two flanking markers. The conditional probability of each allele is determined by the recombination fractions r₁ and r₂ between markers and QTL. Note that at least one flanking marker is required to be informative in order to infer QTL allele. The bottom table shows the formula used to calculate QTL allelic segregation probability based on individual DH population and a consensus map. In practice, r₁ and r₂ are provided by the consensus map.

FIG. 3 represents the mapping population used in network population mapping in Example 3.

FIG. 4 represents a flow chart for a nested population mapping process.

### DETAILED DESCRIPTION OF THE INVENTION

### Overview

Traditional QTL mapping approaches typically involve detection of QTL within a population derived from a single biparental cross. Thus, the genetic diversity in most studies is narrow when compared to that available within the species of interest. Typical breeding programs involve complex mating designs involving multiple inbred lines. Combining information from multiple crosses from diverse parental material can increase the statistical power of QTL detection and improve the precision of the estimation of QTL locations and effects (Rebai & Goffinet, 1993, Theor. Appl. Genet. 86: 1014-1022; Muranty, 1996, Heredity 76: 156-165).

Muranty (1996, Heredity 76:156-165) and Xu (1998, Genetics 148:517-524) describe nested population mapping. In this case, QTL effects are nested (in the statistical sense) within populations and the number of parameters to be estimated increases with the number of populations. However, the lack of connections between populations does not allow a global comparison of the effects of all QTL alleles segregating in the different populations. An alternative approach, described by Blanc et al. ((2006) Theor Appl Genet 113:206-224), is to develop connected populations (common parents among populations). In such an analysis, the effects of alleles segregating are estimated simultaneously, which facilitates a global comparison of QTLs. However, these studies only describe association mapping using connections at the parental level.

Provided herein is a novel approach (referred to as “network population mapping” or “NPM”) for identifying or validating QTL in a mapping population. The methods exploit the shared allelic information (or “haplotypes”) between the connected populations for QTL mapping. For the purposes of the present invention, the terms “haplotype” and “allele” are used interchangeably. Thus, a haplotype may refer to a single allele or may refer to a combination of alleles at multiple loci that are transmitted together on the same chromosome. Likewise, an allele may refer to a single genetic locus or multiple genetic loci on the same chromosome.

The methods are useful for detecting an association between a haplotype and a trait of interest across multiple populations, and involve grouping the members of the multiple diverse populations into “networks” according to the shared haplotype of one or more known genetic markers present in that population. Two or more members of a networked population have a “shared haplotype” when each member of the network possesses the same haplotype form (e.g., the same genetic sequence at a marker locus). This shared haplotype may relate to an individual marker position (e.g., a single SNP), or may comprise multiple marker positions as described elsewhere herein (e.g., within intervals between markers). Thus, individual members of a population are connected at the haplotype level in the population.

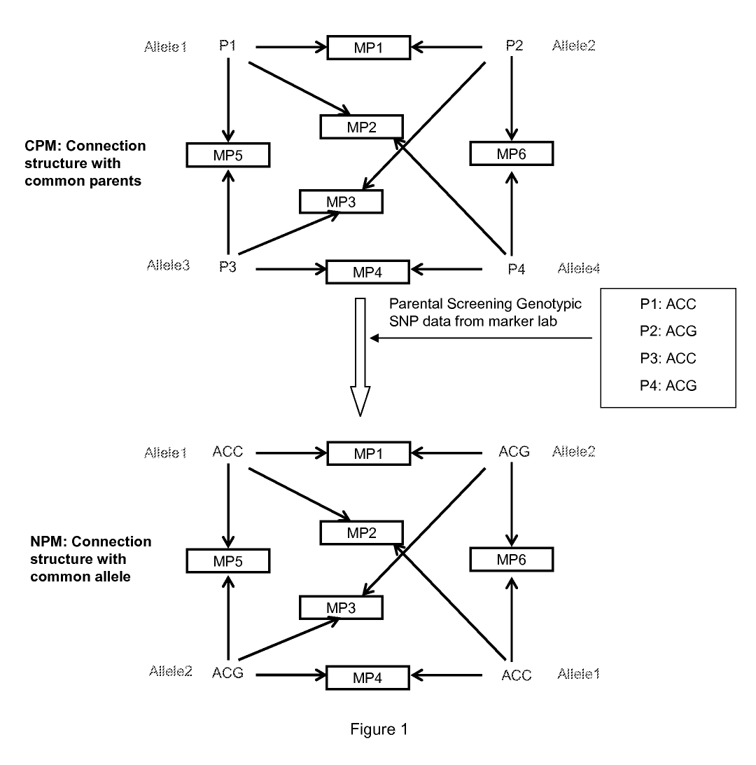

This utilization of shared haplotype information (rather than shared parental information as in connected population mapping) results in increased QTL detection power, thus reducing the overall number of crosses necessary for QTL detection. For example, a population derived from four different parents may have fewer than four different haplotypes at a particular marker locus (see the exemplary population shown in FIG. 1, bottom panel, where there are only two unique alleles measured in the four different parents). By accounting for shared haplotypes within the population (parents and progeny), the number of different groups for QTL analysis is decreased (where a group is defined as having a particular haplotype), and the number of replicates for each group is increased. Thus, where the number of unique alleles is fewer than the number of different parents in a population, the QTL detection power using NPM is higher than with CPM. The methods disclosed herein also provide a means for tracing the actual transition of an allele from parents to their offspring.

The methods further comprise a means for estimating and ranking the effects of multiple alleles across the mapping population, thus allowing breeders to utilize and combine all favorable alleles existing in the multiple connected populations. Detection of QTLs across multiple connected populations also helps provide statistical validation of any QTLs identified in individual biparental QTL mapping.

The methods of the invention involve testing for an association between a marker (or an allelic variant thereof) and a trait of interest. For the purposes of the present invention, a “genetic marker” or a “marker” is intended for a gene or genetic element, or a chromosomal region between two flanking genetic elements (e.g., the interval between two genetic loci) that is being tested for the association. “Allelic variant” refers to the individual alleles (or “haplotypes”) present at a given marker locus. The marker may be an ortholog of a gene known or suspected to be associated with the trait of interest in a different species. As used herein, the term “associated with” in connection with a relationship between a marker (e.g., SNP, haplotype, insertion/deletion, tandem repeat, etc.) and a phenotype refers to a statistically significant dependence of marker frequency with respect to a quantitative scale or qualitative gradation of the phenotype. A marker “positively” correlates with a trait when it is linked to it and when presence of the marker is an indicator that the desired trait or trait form will occur in an organism comprising the marker. A marker negatively correlates with a trait when it is linked to it and when presence of the marker is an indicator that a desired trait or trait form will not occur in an organism comprising the gene. For the purposes of the present invention, the term “marker” refers to any genetic element that is being tested for an association with a trait of interest, and does not necessarily mean that the marker is positively or negatively correlated with the trait of interest.

Thus, a marker is associated with a trait of interest when the genotype of the marker and the trait phenotypes are found together in the progeny of an organism more often than if the genotypes and trait phenotypes segregated separately. The phrase “phenotypic trait” refers to the appearance or other characteristic of an organism, e.g., a plant or animal, resulting from the interaction of its genome with the environment. The term “phenotype” refers to any visible, detectable or otherwise measurable property of an organism. The term “genotype” refers to the genetic constitution of an organism. This may be considered in total, or with respect to the alleles of a single gene, i.e. at a given genetic locus.

In some embodiments, the markers are directly attributable to the phenotypic trait. For example, a genetic element directly attributable to starch accumulation in a plant may be a gene or genetic element directly involved in plant starch metabolism. Alternatively, the marker may be found within a genetic locus associated with the phenotypic trait of interest. A “locus” is a chromosomal region where a polymorphic nucleic acid, trait determinant, gene or marker is located. Thus, for example, a “gene locus” is a specific chromosome location in the genome of a species where a specific gene or genetic element can be found. The marker may also be a known or mapped genetic marker. In various embodiments, the marker identified or validated using the methods disclosed herein may be associated with a quantitative trait locus (QTL). The term “quantitative trait locus” or “QTL” refers to a polymorphic genetic locus with at least two alleles that differentially affect the expression of a phenotypic trait in at least one genetic background.

In some aspects, the markers identified or validated using the methods described herein are linked or closely linked to QTL markers. The phrase “closely linked,” in the present application, means that recombination between two linked loci occurs with a frequency of equal to or less than about 10% (i.e., are separated on a genetic map by not more than 10 cM). In other words, the closely linked loci co-segregate at least 90% of the time. Marker loci are especially useful in the present invention when they demonstrate a significant probability of co-segregation (linkage) with a desired trait. In some aspects, these markers can be termed linked QTL markers.

The methods disclosed herein incorporate a variety of statistical tests and models which may not be explicitly described herein. A thorough description of standard statistical tests can be found in basic textbooks on statistics such as, for example, Dixon, W. J. et al., Introduction to Statistical Analysis, New York, McGraw-Hill (1969) or Steel R. G. D. et al., Principles and Procedures of Statistics: with Special Reference to the Biological Sciences, New York, McGraw-Hill (1960). There are also a number of software programs for statistical analysis that are known to one skilled in the art.

### Population of Interest

The methods disclosed herein are useful for evaluating an association between a marker (or an individual marker haplotype) and a trait of interest across multiple populations. Members of the population are linked according to the particular haplotype shared at one or more polymorphic loci. Thus, individual members of a networked population are grouped for QTL analysis according to the shared haplotypes at a given locus or loci. The genetic region surrounding or within this locus can be evaluated for the presence of a QTL.

The methods provided herein are useful for evaluating an association between a marker and a trait of interest in any connected population. The term “population” or “population of organisms” indicates a group of organisms of the same species, for example, from which samples are taken for evaluation, and/or from which individual members are selected for breeding purposes. In various embodiments, at least one organism, a plurality of organisms, or substantially all of the organisms in the population exhibit a measurable level of the trait of interest. Any number of parents may be used in the mapping population. A particular advantage of the NPM approach described herein compared to the CPM approach is that the actual number of haplotypes for a particular marker is determined by genotyping the markers in all parents in NPM, and members of the population is grouped according to shared haplotypes. In CPM, each parent is assumed to have a distinct allele, thus members of the population are grouped according to shared parents. Thus, the more parents there are in the mapping population, the more complex the CPM analysis becomes due to the assumption of distinct haplotypes for each parent. For example, in CPM, a mapping population of four parents is assumed to have four different haplotypes at a marker locus, a population of six parents is assumed to have six different haplotypes etc. In NPM, the number of different haplotypes is measured, so the number of distinct haplotypes in the population may be lower than the number of parents.

The population members from which the markers are assessed need not be identical to the population members ultimately selected for breeding to obtain progeny, e.g., progeny used for subsequent cycles of analysis. While the methods disclosed herein are exemplified and described primarily using plant populations, the methods are equally applicable to animal populations, for example, humans and non-human animals, such as laboratory animals, domesticated livestock, companion animals, etc.

In some embodiments, the population involves an arbitrary mating design derived from the crosses of multiple inbred lines. In various aspects, the population comprises or consists of a full or partial diallel mating scheme (see, for example, FIG. 1). In some aspects, the parents in the diallel cross are inbreds. As used herein, the term “inbred” means a line that has been bred for genetic homogeneity. Without limitation, examples of breeding methods to derive inbreds include pedigree breeding, recurrent selection, single-seed descent, backcrossing, and doubled haploids. A variety of cross populations can be derived from multiple inbred lines, ranging from a group of independent or related F2 or backcross populations to complicated multiple-generation cross populations with high degree of inbreeding.

In embodiments of the invention, the organism population, such as a plant population, comprises or consists of a population resulting from crosses between one or more founder lines (or progeny thereof) and a single common parent line. In various embodiments, the single common parent line is a tester line. The phrase “tester line” refers to a line that is unrelated to and genetically different from a set of lines to which it is crossed. Using a tester parent in a sexual cross allows one of skill to determine the association of phenotypic trait with expression of quantitative trait loci in a hybrid combination. The phrase “hybrid combination” refers to the process of crossing a single tester parent to multiple lines. The purpose of producing such crosses is to evaluate the ability of the lines to produce desirable phenotypes in hybrid progeny derived from the line by the tester cross.

The progeny of any cross may undergo multiple rounds of “selfing” to generate a population segregating for all genes in a Mendelian fashion. The term “crossed” or “cross” in the context of this invention means the fusion of gametes via pollination to produce progeny (e.g., cells, seeds or plants). The term encompasses both sexual crosses (e.g., the pollination of one plant by another, or the fertilization of one gamete by another) and selfing (e.g., self-pollination, e.g., when the pollen and ovule are from the same plant). The phrase “hybrid” refers to organisms which result from a cross between genetically divergent individuals. The term “lines” in the context of this invention refers to a family of related plants derived by crossing parental lines to derive segregating progeny from that cross. The segregating progeny are then selfed to derive inbred lines. The term “progeny” refers to the descendants of a particular organism (e.g., self crossed plants) or pair of organisms (e.g., through sexual crossing). The descendants can be, for example, of the F₁, the F₂ or any subsequent generation.

The methods disclosed herein further encompass a hybrid cross between a tester line and an elite line. An “elite line” or “elite strain” is an agronomically superior line that has resulted from many cycles of breeding and selection for superior agronomic performance. In contrast, an “exotic strain” or an “exotic germplasm” is a strain or germplasm derived from an organism not belonging to an available elite line or strain of germplasm. Numerous elite lines are available and known to those of skill in the art of breeding. An “elite population” is an assortment of elite individuals or lines that can be used to represent the state of the art in terms of agronomically superior genotypes of a given species. Similarly, an “elite germplasm” or elite strain of germplasm is an agronomically superior germplasm, typically derived from and/or capable of giving rise to an organism with superior agronomic performance. The term “germplasm” refers to genetic material of or from an individual (e.g., a plant or animal), a group of individuals (e.g., a plant line, variety or family), or a clone derived from a line, variety, species, or culture. The germplasm can be part of an organism or cell, or can be separate from the organism or cell. In general, germplasm provides genetic material with a specific molecular makeup that provides a physical foundation for some or all of the hereditary qualities of an organism or cell culture.

In some instances, a population may include parental organisms as well as one or more progeny derived from the parental organisms. In some instances, a population includes members derived from two or more crosses involving the same or different parents. The population may consist of recombinant inbred lines, backcross lines, testcross lines, and the like.

Backcross populations (e.g., generated from a cross between a successful variety (recurrent parent) and another variety (donor parent) carrying a trait not present in the former) can be utilized as a mapping population. In another embodiment, the population consists of inbred plants grouped into pedigrees according to common parents. A “pedigree structure” defines the relationship between a descendant and each ancestor that gave rise to that descendant. A pedigree structure can span one or more generations, describing relationships between the descendant and its parents, grand parents, great-grand parents, etc. The methods of the invention are useful for evaluating an association between a marker and a trait of interest across a single or multiple pedigrees. The connection between the pedigrees is made through haplotypes at one or more genetic marker positions within the population.

The methods of the present invention are applicable to essentially any population or species, particularly plant species. Preferred plants include agronomically and horticulturally important species including, for example, crops producing edible flowers such as cauliflower (Brassica oleracea), artichoke (Cynara scolvmus), and safflower (Carthamus, e.g. tinctorius); fruits such as apple (Malus, e.g. domesticus), banana (Musa, e.g. acuminata), berries (such as the currant, Ribes, e.g. rubrum), cherries (such as the sweet cherry, Prunus, e.g. avium), cucumber (Cucumis, e.g. sativus), grape (Vitis, e.g. vinifera), lemon (Citrus limon), melon (Cucumis melo), nuts (such as the walnut, Juglans, e.g. regia; peanut, Arachis hypoaeae), orange (Citrus, e.g. maxima), peach (Prunus, e.g. persica), pear (Pyra, e.g. communis), pepper (Solanum, e.g. capsicum), plum (Prunus, e.g. domestica), strawberry (Fragaria, e.g. moschata), tomato (Lycopersicon, e.g. esculentum); leafs, such as alfalfa (Medicago, e.g. sativa), sugar cane (Saccharum), cabbages (such as Brassica oleracea), endive (Cichoreum, e.g. endivia), leek (Allium, e.g. porrum), lettuce (Lactuca, e.g. sativa), spinach (Spinacia e.g. oleraceae), tobacco (Nicotiana, e.g. tabacum); roots, such as arrowroot (Maranta, e.g. arundinacea), beet (Beta, e.g. vulgaris), carrot (Daucus, e.g. carota), cassava (Manihot, e.g. esculenta), turnip (Brassica, e.g. rapa), radish (Raphanus, e.g. sativus) yam (Dioscorea, e.g. esculenta), sweet potato (Ipomoea batatas); seeds, such as bean (Phaseolus, e.g. vulgaris), pea (Pisum, e.g. sativum), soybean (Glycine, e.g. max), wheat (Triticum, e.g. aestivum), barley (Hordeum, e.g. vulgare), corn (Zea, e.g. mays), rice (Oryza, e.g. sativa); grasses, such as Miscanthus grass (Miscanthus, e.g., giganteus) and switchgrass (Panicum, e.g. virgatum); trees such as poplar (Populus, e.g. tremula), pine (Pinus); shrubs, such as cotton (e.g., Gossypium hirsutum); and tubers, such as kohlrabi (Brassica, e.g. oleraceae), potato (Solanum, e.g. tuberosum), and the like. The variety associated with any given population can be a transgenic variety, a non-transgenic variety, or any genetically modified variety. Alternatively, plants of a given species naturally occurring in the wild can also be used.

### Genetic Markers

Although specific DNA sequences which encode proteins are generally well-conserved within a species, other regions of DNA (typically non-coding) tend to accumulate polymorphism, and therefore, can be variable between individuals of the same species. Such regions provide the basis for numerous polymorphic molecular genetic markers.

Following generation or selection of one or more populations in the methods disclosed herein, a genotypic value for a plurality of markers is obtained for a plurality of members of the population(s). Members of the mapping population are grouped for QTL analysis by shared haplotypes at one or more marker loci. The genotypic value corresponds to the quantitative or qualitative measure of the genetic marker. The term “marker” refers to an identifiable DNA sequence which is variable (polymorphic) for different individuals within a population, and facilitates the study of inheritance of a trait or a gene. As discussed supra, the marker can be any genetic element that is being tested for an association. A marker at the DNA sequence level is linked to a specific chromosomal location unique to an individual's genotype and inherited in a predictable manner. For each member of the population, haplotype information is collected for a plurality of marker loci. Members are grouped according to shared haplotypes at a particular marker locus or loci and screened for QTL by evaluating the association of the chromosomal region within or surrounding the marker locus, or the chromosomal region flanked by two or more marker loci, and the trait of interest. This association is measured for each haplotype at each genetic marker being evaluated in the population, and the effects of each haplotype on the trait of interest can be ranked in ascending or descending order.

The genetic marker is typically a sequence of DNA that has a specific location on a chromosome that can be measured in a laboratory. The term “genetic marker” can also be used to refer to, e.g., a cDNA and/or an mRNA encoded by a genomic sequence, as well as to that genomic sequence. To be useful, a marker needs to have two or more different haplotypes represented in the population. It will be recognized by one of skill in the art that any given population may have multiple different haplotypes for a particular marker represented in that population. Markers can be either direct, that is, located within the gene or locus of interest, or indirect, that is closely linked with the gene or locus of interest (presumably due to a location which is proximate to, but not inside the gene or locus of interest). Moreover, markers can also include sequences which either do or do not modify the amino acid sequence encoded by the gene in which it is located.

In general, any differentially inherited polymorphic trait (including nucleic acid polymorphism) that segregates among progeny is a potential marker. The term “polymorphism” refers to the presence in a population of two or more allelic variants. The term “allele” or “allelic” refers to one member of a pair or series of different forms of a gene or genetic element; in the case of a SNP this is the actual nucleotide which is present; for a SSR, it is the number of repeat sequences; for a peptide sequence, it is the actual amino acid present. For the purposes of the present invention, the terms “allele” and “haplotype” are used interchangeably. Thus, an allele may represent a single nucleotide position (such as a SNP), or may represent the combination of two or more positions present on the same chromosome and inherited together. An “associated allele” refers to an allele at a polymorphic locus which is associated with a particular phenotype of interest. Such allelic variants include sequence variation at a single base, for example a single nucleotide polymorphism (SNP). A polymorphism can be a single nucleotide difference present at a locus, or can be an insertion or deletion of one, a few or many consecutive nucleotides. It will be recognized that while the methods of the invention are exemplified primarily by the detection of SNPs, these methods or others known in the art can similarly be used to identify other types of polymorphisms, which typically involve more than one nucleotide.

The genomic variability can be of any origin, for example, insertions, deletions, duplications, repetitive elements, point mutations, recombination events, or the presence and sequence of transposable elements. The marker may be measured directly as a DNA sequence polymorphism, such as a single nucleotide polymorphism (SNP), restriction fragment length polymorphism (RFLP) or short tandem repeat (STR), or indirectly as a DNA sequence variant, such as a single-strand conformation polymorphism (SSCP). A marker can also be a variant at the level of a DNA-derived product, such as an RNA polymorphism/abundance, a protein polymorphism or a cell metabolite polymorphism, or any other biological characteristic which has a direct relationship with the underlying DNA variant or gene product.

Two types of markers are frequently used in mapping and marker assisted breeding protocols, namely simple sequence repeat (SSR, also known as microsatellite) markers, and single nucleotide polymorphism (SNP) markers. The term SSR refers generally to any type of molecular heterogeneity that results in length variability, and most typically is a short (up to several hundred base pairs) segment of DNA that consists of multiple tandem repeats of a two or three base-pair sequence. These repeated sequences result in highly polymorphic DNA regions of variable length due to poor replication fidelity, e.g., caused by polymerase slippage. SSRs appear to be randomly dispersed through the genome and are generally flanked by conserved regions. SSR markers can also be derived from RNA sequences (in the form of a cDNA, a partial cDNA or an EST) as well as genomic material.

In one embodiment, the molecular marker is a single nucleotide polymorphism. Various techniques have been developed for the detection of SNPs, including allele specific hybridization (ASH; see, e.g., Coryell et al., (1999) Theor. Appl. Genet., 98:690-696). Additional types of molecular markers are also widely used, including but not limited to expressed sequence tags (ESTs) and SSR markers derived from EST sequences, amplified fragment length polymorphism (AFLP), randomly amplified polymorphic DNA (RAPD) and isozyme markers. A wide range of protocols are known to one of skill in the art for detecting this variability, and these protocols are frequently specific for the type of polymorphism they are designed to detect. For example, PCR amplification, single-strand conformation polymorphisms (SSCP) and self-sustained sequence replication (3SR; see Chan and Fox, Reviews in Medical Microbiology 10:185-196).

DNA for genotyping and association analysis may be collected and screened in any convenient tissue of an organism of interest, for example from cells, seed or tissues from which plants may be grown, or plant parts, such as leaves, stems, pollen, or cells, that can be cultured into a whole plant. In some embodiments, genotype data is taken from tissues that have been associated with the trait under study. In some embodiments of the present invention, genotype data is measured from multiple tissues of each organism under study. A sufficient number of cells are obtained to provide a sufficient amount of sample for analysis, although only a minimal sample size will be needed where scoring is by amplification of chromosomal regions or nucleic acids. The DNA, RNA, or protein can be isolated from the cell sample by standard nucleic acid isolation techniques known to those skilled in the art.

In one embodiment, the markers correspond to the values obtained for essentially all, or all, of the SNPs of a high-density, whole genome SNP map. This approach has the advantage over traditional approaches in that, since it encompasses the whole genome, it identifies potential interactions of genomic products expressed from genes located anywhere on the genome without requiring preexisting knowledge regarding a possible interaction between the genomic products. An example of a high-density, whole genome SNP map is a map of at least about 1 SNP per 10,000 kb, at least 1 SNP per 500 kb or about 10 SNPs per 500 kb, or at least about 25 SNPs or more per 500 kb. Definitions of densities of markers may change across the genome and are determined by the degree of linkage disequilibrium within a genome region.

Additionally, a number of genetic marker screening platforms are now commercially available, and can be used to obtain the genetic marker data required for the process of the present methods. In many instances, these platforms can take the form of genetic marker testing arrays (microarrays), which allow the simultaneous testing of many thousands of genetic markers. For example, these arrays can test genetic markers in numbers of greater than 1,000, greater than 1,500, greater than 2,500, greater than 5,000, greater than 10,000, greater than 15,000, greater than 20,000, greater than 25,000, greater than 30,000, greater than 35,000, greater than 40,000, greater than 45,000, greater than 50,000 or greater than 100,000, greater than 250,000, greater than 500,000, greater than 1,000,000, greater than 5,000,000, greater than 10,000,000 or greater than 15,000,000. Examples of such a commercially available product for are those marketed by Affymetrix Inc ((www.affymetrix.com)) or Illumina (www.illumina.com). In one embodiment, the genotypic value is obtained from at least 2 genetic markers.

It will be appreciated that, due to the nature of such information, a filtering or preprocessing of the data may be required, i.e., quality control of the data. For example, marker data may be excluded according to a particular criteria (e.g., data duplication or low frequency; see, for example Zenger et. al (2007) Anim Genet. 38(1):7-14). Examples of such filtering are described below, although other methods of filtering the data as would be appreciated by the skilled artisan may also be employed to obtain a working data set on which the marker association is determined.

In one embodiment, marker data is excluded from the analysis where the allele frequency of a particular marker is less than about 0.01, or less than about 0.05. “Allele frequency” or “marker allele frequency” (MAF) refers to the frequency (proportion or percentage) at which an allele is present at a locus within an individual, within a line, or within a population of lines. For example, for an allele “A,” diploid individuals of genotype “AA,” “Aa,” or “aa” have allele frequencies of 1.0, 0.5, or 0.0, respectively. One can estimate the allele frequency within a line by averaging the allele frequencies of a sample of individuals from that line. Similarly, one can calculate the allele frequency within a population of lines by averaging the allele frequencies of lines that make up the population. For a population with a finite number of individuals or lines, an allele frequency can be expressed as a count of individuals or lines (or any other specified grouping) containing the allele.

In various embodiments, the markers evaluated in the methods disclosed herein may be random markers as described above, or may be markers or genetic elements that have been shown or are suspected to be associated with the trait of interest in a different plant species. A large number of positively associated markers for various species are known in the art and can be validated in different species using the methods disclosed herein. For example, a group of markers that has been identified based on their molecular functions and/or performances in corn may be tested in soybean. Thus, the models described herein are useful for validating the effects of these markers in a different plant species. When evaluating a set of markers, generally random markers having no known association will also be included in the analysis.

### Association Analysis

Genetics data have been used in the field of trait analysis in order to attempt to identify the genes that affect such traits. A key development in such pursuits has been the development of large collections of molecular/genetic markers, which can be used to construct detailed genetic maps of species. The objective of genetic mapping is to identify simply inherited markers in close proximity to genetic factors affecting quantitative traits, that is, QTL. This localization relies on processes that create a statistical association between marker and QTL alleles and processes that selectively reduce that association as a function of the marker distance from the QTL.

The methods of the present invention encompass novel strategies for identifying or validating the association of a marker and a trait of interest across multiple connected populations. Members of the population are grouped for association analysis based on the presence of common alleles, or haplotype alleles, at one or more genetic marker loci. Marker data at regular intervals across the genome under study or in gene regions of interest are used to monitor segregation or detect associations in a population of interest. In some embodiments, these regularly defined intervals are defined in Morgans or, more typically, centimorgans (cM). A Morgan is a unit that expresses the genetic distance between markers on a chromosome. A Morgan is defined as the distance on a chromosome in which one recombination event is expected to occur per gamete per generation. In some embodiments, each regularly defined interval is less than 100 cM. In other embodiments, each regularly defined interval is less than 10 cM, less than 5 cM, less than 2.5 cM, less than 2 cM, less than 1.5 cM, or less than 1 cM.

In order to determine which markers will be used for genotyping in each biparental mapping population, parental marker screening (PMS) is performed. The main purpose of PMS is to check the polymorphism of a large set of markers among parents based on a consensus map. With PMS, the SNP haplotype is used to characterize marker genotype among parents.

Where the genotype is homozygous for each parent, one genotype stands for one haplotype. In many screening programs, several SNP assays are performed within each locus, and these assays form haplotypes. In the context of NPM, each haplotype is considered as a unique allele.

PMS provides the haplotype information of each locus for the parents of NPM. For instance, the haplotypes AGC, ACG, and TCC may be observed for parent 1, 2 and 3, respectively, at a locus. This means that these three parents carry three different alleles at the locus. In another example, parent 1, 2, and 3 may carry alleles AGC, AGC and TCC, respectively. Parent 1 and 2 carry the same allele AGC, and parent 3 has the different allele TCC. Thus, haplotype allelic information can be obtained by PMS, and a set of polymorphic markers can be selected for association analysis based on this screening.

Models For Network Population Mapping

Several types of known statistical analyses can be used to infer marker/trait association from the phenotype/genotype data, but the central idea of the present invention is to detect markers, i.e., polymorphisms, for which alternative genotypes have significantly different average phenotypes. For example, if a given marker locus A has three alternative genotypes (AA, Aa and aa), and if those three classes of individuals have significantly different phenotypes, then one infers that locus A (or “a”) is associated with the trait. The significance of differences in phenotype may be tested by several types of standard statistical tests such as linear regression of marker genotypes on phenotype or analysis of variance (ANOVA). A genetic map is created by placing genetic markers in genetic (linear) map order so that the positional relationships between markers are understood.

In the present invention, the shared allelic information between connected populations is utilized to evaluate allele-specific associations between a marker and a trait of interest. Members of the network share a common allele at one or more marker loci, and can be used to identify or validate QTL within the chromosomal region within, surrounding or flanked by the marker loci.

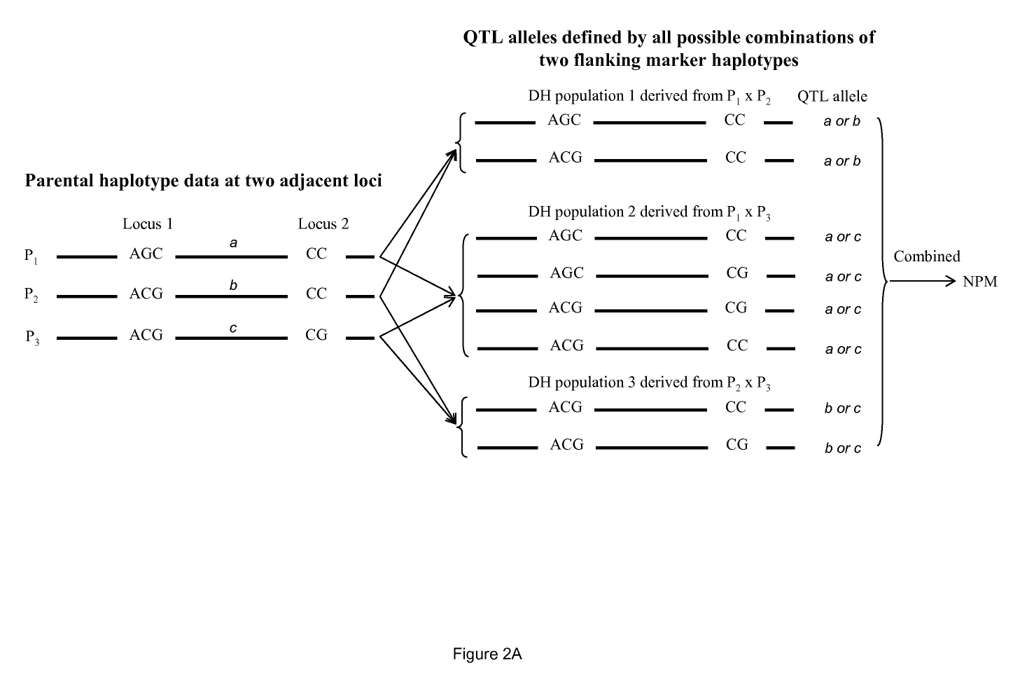

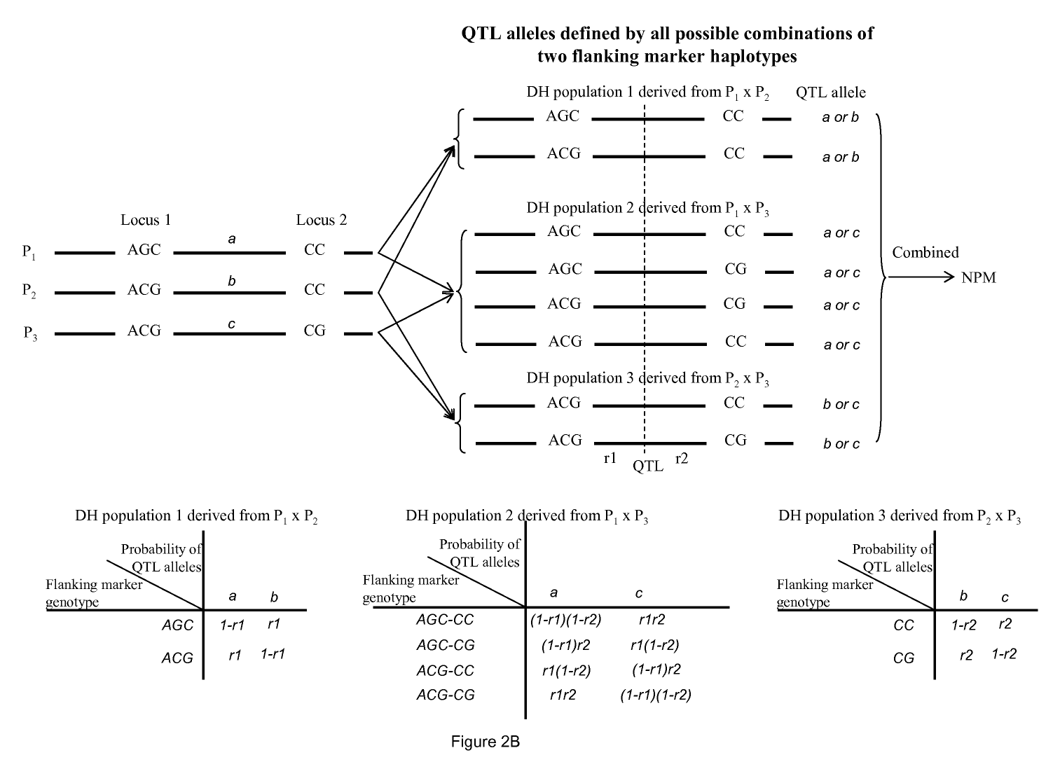

In various embodiments, the association model useful herein comprises a means for evaluating whether a particular haplotype in question is present in the networked population. When using interval mapping approaches, this variable is unobservable but may be inferred by the genotypes of two flanking markers (Lander and Botstein 1989; Haley and Knott 1992). When evaluating the phenotypic value of a test crossed hybrid (or inbred), only additive effects of a QTL are considered, since the dominant effect cannot be tested. Thus, this variable may be inferred conditional on the genotypes of its two flanking markers. In a mapping population, it is possible to have multiple alleles (e.g., alleles 1, 2, 3 . . . n) at each locus. Thus, the conditional probability of each haplotype coming from a specific population is computed based on a consensus map as described elsewhere herein (FIG. 2B).

The association model useful for NPM further comprises a means for measuring the additive effect of the allele in question. In various embodiments, the allelic effect of a particular allele is treated as a random factor in the model rather than as a fixed effect as described in the art. Specifically, the allelic effect is assumed to follow a normal distribution with mean zero and genetic variance σ_{g}². This assumption is made so that the BLUP (best linear unbiased estimate) can be obtained for each allele. “BLUP” refers to a statistical technique which is widely used to provide prediction of genetic merit (Henderson C. R. (1973) Sire Evaluation and Genetic Trends. in Proc. Anim. Breed. Genet. Symp. Am. Soc. Anim. Sci. and Am. Dairy Sci. Assoc. Champaign, Ill., 10-41). BLUP can be performed, by those of ordinary skill in the art, using any of the various commercially available computer programs that are used for genetic evaluation of an individual or a population. Standard software packages that are publicly available can be used to perform BLUP (e.g. “BLUPF90” on the internet at nce.ads.uga.edu/.about.ignacy/newprograms.html).

Another advantage to treating the allelic effect as a random factor in the model is in overcoming the problem of hypothesis testing. Generally speaking, when scanning the whole genome using a specific interval such as 1 or 2 cM, it is possible to have different number of alleles at each tested position. If the allelic effect is treated as a fixed effect, the number of degree freedom may vary test by test, and it is difficult to apply a genome-wide LOD threshold to test the significance of allelic effect along the whole genome. Methods for testing allelic effect using genome-wide LOD threshold are discussed elsewhere herein.

In yet another embodiment, the association model comprises a means for accounting for the influences of different genetic backgrounds from individual populations. In some embodiments, this effect is assumed to be a fixed effect.

NPM provides increased QTL detection power and mapping resolution in contrast to other connected population mapping methods. In the case of CPM, the basic assumption is that every parent involved in a connected population has a unique haplotype at every polymorphic marker locus used in the analysis, but this assumption does not necessarily hold true in populations with shared ancestry, especially breeding populations. For example, in a connected population with 6 parents, CPM methods assume there will be six haplotypes for each polymorphic marker locus, whereas the actual number of observed haplotypes might vary from 2 to 6. NPM methods utilize the actual number of different haplotypes. In the example described above, if there are only 3 haplotypes at a marker locus, the power for QTL detection using NPM is twice that of the power for QTL detection using CPM because each haplotype in CPM will only have half the number of replicates compared to the replicates for each haplotype in NPM. The effects of each haplotype may be estimated by BLUP approach. This approach makes it possible to obtain a global ranking of haplotypes responsible for the trait of interest across all the connected populations. The estimating and ranking of allelic effects for haplotypes are particularly useful for marker assisted selection based on the connected populations.

In various embodiments of the present invention, a simple interval mapping (SIM) approach is used to evaluate haplotype-specific associations of a marker and a trait of interest. All SIM procedures search for a single “target QTL” at positions throughout a mapped genome. The novel SIM approach described herein allows for estimating and ranking of multiple haplotypes (or “alleles”) at a marker locus. A novel SIM model useful in the methods disclosed herein is:

yᵢⱼ=μ+zᵢⱼa^{q}+gᵢ+eᵢⱼ (model 1);

where yᵢⱼ is the trait value of the test crossed hybrid (or inbred) j in the population i; μ is the overall mean; zᵢⱼ is the indicator variable showing whether the allele q is present in a population; a is the additive effect of the allele q of a QTL; gᵢ is the polygenetic effect of the background i defined by the population i; and eᵢⱼ is the residual term after accounting for QTL and polygenetic effects in the trait data. In the model, the parameter gᵢ is assumed to be a fixed effect, and used to account for the influences of different genetic backgrounds from individual population based on pedigree. The residual eᵢⱼ follows a normal distribution with mean zero and the residual variance σₑ².

In another embodiment of the invention, haplotype-specific associations are detected or validated using composite interval mapping. CIM handles multiple QTLs by incorporating multi locus marker information from organisms by modifying standard interval mapping to include additional markers as cofactors for analysis. In these methods, one performs interval mapping using a subset of marker loci as covariates. These markers serve as proxies for other QTLs to increase the resolution of interval mapping, by accounting for linked QTLs and reducing the residual variation.

A novel CIM model useful in the methods of the present invention includes: Now consider the linear model

yᵢⱼ=μ+zᵢⱼa^{q}+Σ(k=1, c)xᵢⱼₖbₖ+gᵢ+eᵢⱼ (model 2);

where xᵢⱼₖ is the genotype of the cofactor marker k (k=1, 2, . . . , c) of the line j in the population i and bₖ is the effect of the marker k. The notations of other terms in model 2 are same as those in model 1.

The only difference between models 1 and 2 is the inclusion of cofactor markers in the latter. These cofactors are used to absorb the influences from other QTL, and then improve the precision of parameter estimation. In various embodiments of the present invention, SIM can be used in combination with CIM to identify QTL.

The method used for selecting cofactors is stepwise regression based on the model:

yᵢⱼ=μ+Σ(k=1, c)xᵢⱼₖbₖ+gᵢ+eᵢⱼ (model 3).

Note that the regression term gᵢ enters the model before choosing any cofactors, and it is always retained in the model with the selection of cofactors. In some embodiments, the significance level to add a new variable into the model is at least about 0.01 or higher.

Probability Distribution of QTL Genotype

As discussed supra, the unobservable QTL alleles may be inferred from the observed genotypes at marker loci that flank the QTL. The location and identity of these flanking markers can be obtained from a consensus genetic map of the species of interest, and the genotype of these markers can be obtained by parental marker screening. The QTL alleles (i.e., marker) being evaluated are thus within the interval of the flanking markers. This interval-based approach for QTL evaluation differs from the existing connected population mapping approaches described in the art, which all use marker-based approaches. However, it will be understood by one of skill in the art that marker-based association mapping approaches are also useful in the methods disclosed herein.

An interval is defined by the haplotypes of two flanking markers, say, marker m and m+1. Suppose the haplotypes of the marker m for three parents are AGC, ACG, and AGC, and the ones for the marker m+1 are CC, CC, and GG (FIG. 2A). Then, there are three different haplotypes AGC-CC, ACG-CC, and AGC-CG for the interval. Here, these interval haplotypes are used to stand for QTL alleles a, b, and c within the interval.

Based on a consensus map, the computation of probability distribution of QTL alleles a, b and c is conditional upon haplotypes of flanking markers. Specifically, there are three scenarios for two flanking markers m and m+1 (FIG. 2B). In the first scenario, the markers m and m+1 are not polymorphic for a population. For this case, it is assumed that the interval defined by the two markers holds a monomorphic QTL allele in the population. However, the state of the allele derived from two flanking markers can be obtained by PMS for the population. The second scenario is that there is only one marker, say m, which is polymorphic in a population. In this situation, the probability of a QTL genotype is inferred based only on the marker m and the recombination fraction r between QTL and the marker m. In the last scenario, markers m and m+1 are polymorphic in a population, and the probability distribution of QTL genotype may be computed using conventional interval mapping (FIG. 2B).

### Testing QTL Effect

In the present invention, the goal is not simply to detect marker/trait associations, but to estimate the effect of the allele q of a QTL. The genotype/phenotype data are used to calculate for each test position a LOD score (log of likelihood ratio). When the LOD score exceeds a critical threshold value, there is significant evidence for the allelic effect of a QTL at that position on the genetic map (which will fall within an interval between two particular marker loci).

Thus, in the present invention, the allelic effect is measured by calculating an LOD score for each allele at each marker locus. For each trait under study, only the values which exceed the threshold LOD score (based on permutation testing as described infra) are retained for the purpose of locating QTL peaks. This data is then processed using SAS software that scans all chromosomes from top to bottom to identify QTL peaks. In this program, QTL peaks are identified based on the sudden drop in the LOD score that follows a peak.

An interval of about 0.5, about 1, about 1.5, about 2, about 2.5, about 3 or more cM is also scanned for defining the confidence interval (“CI,” e.g., the 90% CI, 95% CI, or greater). The LOD and map position values from these intervals are populated for all of the QTLs detected in the earlier step.

The trait(s) of interest being evaluated are assigned either a positive “+” or a negative “−” sign based on whether a user generally selects for higher values or lower values in the segregating progeny (i.e., whether the desired trait is an increase in a particular phenotypic value (e.g., yield), or a decrease in a particular phenotypic value (e.g., disease presence). These criteria, along with the absolute allele effect values of the detected QTLs are then used to develop a ranking order for both QTLs and their allelic effects.

For each trait of interest, each QTL detected across all chromosomes is ranked based on the sum value of the product of the LOD value and the absolute maximum additive value observed for all alleles tested at that QTL position. For allele ranking, if the trait under study is positive, then the allele with the highest effect is considered as the most favorable, or if the trait under study is negative then the allele with the smallest effect on trait phenotype is considered the most favorable. At each of the QTL peaks, multiple allele effects are sorted either in descending order (for positive traits) or ascending order (for negative traits). Each allele is assigned a ranking order number based on this sorting.

Hypothesis Testing

To determine whether an association exists between a marker and a phenotypic trait of interest, hypothesis testing is performed. The hypotheses to test QTL effect can be formulated as H₀: σ_{g}²=0 and H₁: σ_{g}²≠0. Then the likelihood ratio (LR) can be obtained. The likelihood ratio is the ratio of the maximum probability of a result under two different hypotheses. A likelihood-ratio test is a statistical test for making a decision between two hypotheses based on the value of this ratio. Being a function of the data x, the LR is therefore a statistic. The likelihood-ratio test rejects the null hypothesis if the value of this statistic is too small. How small is too small depends on the significance level of the test, i.e., on what probability of Type I error is considered tolerable (“Type I” errors consist of the rejection of a null hypothesis that is true).

Lower values of the likelihood ratio mean that the observed result is less likely to occur under the null hypothesis. Higher values mean that the observed result is more likely to occur under the null hypothesis. The LR can be obtained from the regression models as LR=−2(l_{reduced}−l_{full}), where l_{reduced} is the log likelihood of the reduced model, corresponding to H₀, and l_{full} is that of the full model, corresponding to H₁ (Lander and Botstein 1989).

From the LR, a logarithm of the odds (LOD) score is calculated. A LOD score is a statistical estimate of whether two loci are likely to lie near each other on a chromosome and are therefore likely to be genetically linked. In the present case, a LOD score is a statistical estimate of whether a given position in the genome under study is linked to the quantitative trait corresponding to a given gene. In one embodiment, the LOD score is calculated as LR/(2 ln 10). The LOD score essentially indicates how much more likely the data are to have arisen assuming the presence of a positively-associated QTL versus in its absence. The LOD threshold value for avoiding a false positive with a given confidence, say 95%, depends on the number of markers and the length of the genome. Graphs indicating LOD thresholds are set forth in Lander and Botstein, Genetics, 121:185-199 (1989), and further described by Ars and Moreno-Gonzalez, Plant Breeding, Hayward, Bosemark, Romagosa (eds.) Chapman & Hall, London, pp. 314-331 (1993). To determine the empirical LOD threshold, permutation tests are used.

Permutation Tests

To determine the appropriate LOD threshold for NPM, permutation tests are used because the theoretical probability distribution of LOD is unclear. Permutation tests essentially measure the confidence of the association of the QTL and the trait of interest. One of the most important steps in QTL analysis is to decide on a threshold value for the test statistic. If the threshold is not exceeded, the null hypothesis (no QTL) is accepted. If the threshold is exceeded, the alternate hypothesis (QTL presence) is made. A threshold is usually chosen to give a specific type I error rate (e.g. P=0.05). Permutation involves scrambling the order of the data randomly so that the effects of the parameters are lost. This produces a set of data that represents the null hypothesis. The distribution of the test statistic under the null hypothesis is derived by computing the test statistic in many random permutations of the original data. One can then choose a test statistic that is larger than (e.g.) 95%, 96%, 97%, 98%, or 99% of this distribution.

The permutation method useful in the present invention reshuffles the phenotypic values within each subpopulation without destroying the structure of subpopulations and the correlation between different traits of interest. See, for example, the permutation method described in U.S. patent application Ser. No. 12/367,045, filed Feb. 6, 2009, which is herein incorporated by reference in its entirety.

### Trait of Interest

The methods of the present invention are applicable to any phenotypic trait with an underlying genetic component, i.e., any heritable trait. A “trait” is a characteristic of an organism which manifests itself in a phenotype, and refers to a biological, performance or any other measurable characteristic(s), which can be any entity which can be quantified in, or from, a biological sample or organism, which can then be used either alone or in combination with one or more other quantified entities. A “phenotype” is an outward appearance or other visible characteristic of an organism and refers to one or more trait of an organism.

Many different traits can be inferred by the methods disclosed herein. The phenotype can be observable to the naked eye, or by any other means of evaluation known in the art, e.g., microscopy, biochemical analysis, genomic analysis, an assay for a particular disease resistance, etc. In some cases, a phenotype is directly controlled by a single gene or genetic locus, i.e., a “single gene trait.” In other cases, a phenotype is the result of several genes. A “quantitative trait loci” (QTL) is a genetic domain that is polymorphic and effects a phenotype that can be described in quantitative terms, e.g., height, weight, oil content, days to germination, disease resistance, etc, and, therefore, can be assigned a “phenotypic value” which corresponds to a quantitative value for the phenotypic trait.

For any trait, a “relatively high” characteristic indicates greater than average, and a “relatively low” characteristic indicates less than average. For example “relatively high yield” indicates more abundant plant yield than average yield for a particular plant population. Conversely, “relatively low yield” indicates less abundant yield than average yield for a particular plant population.

In the context of an exemplary plant breeding program, quantitative phenotypes include, yield (e.g., grain yield, silage yield), stress (e.g., mid-season stress, terminal stress, moisture stress, heat stress, etc.) resistance, disease resistance, insect resistance, resistance to density, kernel number, kernel size, ear size, ear number, pod number, number of seeds per pod, maturity, time to flower, heat units to flower, days to flower, root lodging resistance, stalk lodging resistance, ear height, grain moisture content, test weight, starch content, grain composition, starch composition, oil composition, protein composition, nutraceutical content, and the like.

In addition, the following phenotypic values may be correlated with a marker: color, size, shape, skin thickness, pulp density, pigment content, oil deposits, protein content, enzyme activity, lipid content, sugar and starch content, chlorophyll content, minerals, salt content, pungency, aroma and flavor and such other features. For each of these indices, a distribution of parameters is determined for the sample by determining a feature (e.g., weight) associated with each item in the sample, and then measuring mean and standard deviation values from the distribution.

Similarly, the methods are equally applicable to traits which are continuously variable, such as grain yield, height, oil content, response to stress (e.g., terminal or mid-season stress) and the like, or to meristic traits that are multi-categorical, but can be analyzed as if they were continuously variable, such as days to germination, days to flowering or fruiting, and to traits with are distributed in a non-continuous (discontinuous) or discrete manner. However, it is to be understood that analogous or other unique traits may be characterized using the methods described herein, within any organism of interest.

In addition to phenotypes directly assessable by the naked eye, with or without the assistance of one or more manual or automated devices, included, e.g., microscopes, scales, rulers, calipers, etc., many phenotypes can be assessed using biochemical and/or molecular means. For example, oil content, starch content, protein content, nutraceutical content, as well as their constituent components can be assessed, optionally following one or more separation or purification step, using one or more chemical or biochemical assay. Molecular phenotypes, such as metabolite profiles, MAS spectrometry, or expression profiles, either at the protein or RNA level, are also amenable to evaluation according to the methods of the present invention. For example, metabolite profiles, whether small molecule metabolites or large bio-molecules produced by a metabolic pathway, supply valuable information regarding phenotypes of agronomic interest. Such metabolite profiles can be evaluated as direct or indirect measures of a phenotype of interest. Similarly, expression profiles can serve as indirect measures of a phenotype, or can themselves serve directly as the phenotype subject to analysis for purposes of marker correlation. Expression profiles are frequently evaluated at the level of RNA expression products, e.g., in an array format, but may also be evaluated at the protein level using antibodies or other binding proteins.

In addition, in some circumstances it is desirable to employ a mathematical relationship between phenotypic attributes rather than correlating marker information independently with multiple phenotypes of interest. For example, the ultimate goal of a breeding program may be to obtain crop plants which produce high yield under low water, i.e., drought, conditions. Rather than independently correlating markers for yield and resistance to low water conditions, a mathematical indicator of the yield and stability of yield over water conditions can be correlated with markers. Such a mathematical indicator can take on forms including; a statistically derived index value based on weighted contributions of values from a number of individual traits, or a variable that is a component of a crop growth and development model or an ecophysiological model (referred to collectively as crop growth models) of plant trait responses across multiple environmental conditions. These crop growth models are known in the art and have been used to study the effects of genetic variation for plant traits and map QTL for plant trait responses. See references by Hammer et al. 2002. European Journal of Agronomy 18: 15-31, Chapman et al. 2003. Agronomy Journal 95: 99-113, and Reymond et al. 2003. Plant Physiology 131: 664-675.

### Computer-Implemented Methods

The methods described above for evaluating a marker: trait association may be performed, wholly or in part, with the use of a computer program or computer-implemented method.

Computer programs and computer program products of the present invention comprise a computer usable medium having control logic stored therein for causing a computer to execute the algorithms disclosed herein. Computer systems of the present invention comprise a processor, operative to determine, accept, check, and display data, a memory for storing data coupled to said processor, a display device coupled to said processor for displaying data, an input device coupled to said processor for entering external data; and a computer-readable script with at least two modes of operation executable by said processor. A computer-readable script may be a computer program or control logic of a computer program product of an embodiment of the present invention.

It is not critical to the invention that the computer program be written in any particular computer language or to operate on any particular type of computer system or operating system. The computer program may be written, for example, in C++, Java, Perl, Python, Ruby, Pascal, or Basic programming language. It is understood that one may create such a program in one of many different programming languages. In one aspect of this invention, this program is written to operate on a computer utilizing a Linux operating system. In another aspect of this invention, the program is written to operate on a computer utilizing a MS Windows or Mac OS operating system.

It would be understood by one of skill in the art that codes may be performed in any order, or simultaneously, in accordance with the present invention so long as the order follows a logical flow.

### Downstream Use of Positively Associated Markers

The markers identified or validated using the methods disclosed herein may be used for genome-based diagnostic and selection techniques; for tracing progeny of an organism; to determine hybridity, uniformity, and purity of an organism; to identify variation of linked phenotypic traits, mRNA expression traits, or both phenotypic and mRNA expression traits; as genetic markers for constructing genetic linkage maps; to identify individual progeny from a cross wherein the progeny have a desired genetic contribution from a parental donor, recipient parent, or both parental donor and recipient parent; to isolate genomic DNA sequence surrounding a gene-coding or non-coding DNA sequence, for example, but not limited to a promoter or a regulatory sequence; in marker-assisted selection, map-based cloning, hybrid certification, fingerprinting, genotyping and allele specific marker; for transgenic plant development; and, as a marker in an organism of interest.

The primary motivation for developing molecular marker technologies from the point of view of plant breeders has been the possibility to increase breeding efficiency through marker assisted breeding. After positive markers have been identified through the statistical models described above, the corresponding favorable alleles can be used to identify plants that contain the desired genotype at multiple loci and would be expected to transfer the desired genotype along with the desired phenotype to its progeny. A molecular marker allele that demonstrates linkage disequilibrium with a desired phenotypic trait (e.g., a quantitative trait locus, or QTL) provides a useful tool for the selection of a desired trait in a plant population (i.e., marker assisted breeding).

Thus, the present invention also comprises methods for breeding a population of organisms exhibiting a trait of interest. The method comprises identifying a marker that is associated with said trait of interest using the NPM method disclosed herein.

The markers and/or alleles that are identified using these methods are used to select plants and enrich the plant population for individuals that have desired traits. By identifying and selecting a marker allele (or desired alleles from multiple markers) that is optimized for the desired phenotype, the plant breeder is able to rapidly select a desired phenotype by selecting for the optimized allele. Plants comprising the optimized allele can then be crossed with compatible plants (i.e., plants that can be crossed to result in progeny), and the resulting progeny can be screened for the presence of the associated marker.

The presence and/or absence of a particular desired allele in the genome of a plant exhibiting a preferred phenotypic trait is determined by any method known in the art, e.g., RFLP, AFLP, SSR, amplification of variable sequences, and ASH. If the nucleic acids from the plant hybridizes to a probe specific for a desired genetic marker, the plant can be selfed to create a true breeding line with the same genome or it can be introgressed into one or more lines of interest. The term “introgression” refers to the transmission of a desired allele of a genetic locus from one genetic background to another. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., a selected allele of a marker, a QTL, a transgene, or the like. In any case, offspring comprising the desired allele can be repeatedly backcrossed to a line having a desired genetic background and selected for the desired allele, to result in the allele becoming fixed in a selected genetic background. In various embodiments, a combination of favorable alleles can be assembled into a single line.

The marker loci identified or validated using the methods of the present invention can also be used to create a dense genetic map of molecular markers. A “genetic map” is a description of genetic linkage relationships among loci on one or more chromosomes (or linkage groups) within a given species, generally depicted in a diagrammatic or tabular form. “Genetic mapping” is the process of defining the linkage relationships of loci through the use of genetic markers, populations segregating for the markers, and standard genetic principles of recombination frequency. A “genetic map location” is a location on a genetic map relative to surrounding genetic markers on the same linkage group where a specified marker can be found within a given species. In contrast, a physical map of the genome refers to absolute distances (for example, measured in base pairs or isolated and overlapping contiguous genetic fragments, e.g., contigs). A physical map of the genome does not take into account the genetic behavior (e.g., recombination frequencies) between different points on the physical map.

In certain applications it is advantageous to make or clone large nucleic acids to identify nucleic acids more distantly linked to a given marker, or isolate nucleic acids linked to or responsible for QTLs as identified herein. It will be appreciated that a nucleic acid genetically linked to a polymorphic nucleotide sequence optionally resides up to about 50 centimorgans from the polymorphic nucleic acid, although the precise distance will vary depending on the cross-over frequency of the particular chromosomal region. Typical distances from a polymorphic nucleotide are in the range of 1-50 centimorgans, for example, often less than 1 centimorgan, less than about 1-5 centimorgans, about 1-5, 1, 5, 10, 15, 20, 25, 30, 35, 40, 45 or 50 centimorgans, etc.

Many methods of making large recombinant RNA and DNA nucleic acids, including recombinant plasmids, recombinant lambda phage, cosmids, yeast artificial chromosomes (YACs), P1 artificial chromosomes, Bacterial Artificial Chromosomes (BACs), and the like are known. A general introduction to YACs, BACs, PACs and MACs as artificial chromosomes is described in Monaco & Larin, Trends Biotechnol. 12:280-286 (1994). Examples of appropriate cloning techniques for making large nucleic acids, and instructions sufficient to direct persons of skill through many cloning exercises are also found in Berger, Sambrook, and Ausubel, all supra.

In addition, any of the cloning or amplification strategies described herein are useful for creating contigs of overlapping clones, thereby providing overlapping nucleic acids which show the physical relationship at the molecular level for genetically linked nucleic acids. A common example of this strategy is found in whole organism sequencing projects, in which overlapping clones are sequenced to provide the entire sequence of a chromosome. In this procedure, a library of the organism's cDNA or genomic DNA is made according to standard procedures described, e.g., in the references above. Individual clones are isolated and sequenced, and overlapping sequence information is ordered to provide the sequence of the organism.

In various embodiments, the markers tested in the methods disclosed herein are candidate genes, or are polymorphic regions within candidate genes. Once a gene (or set of genes) is determined to be associated with a trait of interest in a particular organism, the gene(s) can be transformed into the organism to obtain the phenotypic trait of interest. The gene can be incorporated into an expression construct and operably linked to a promoter functional in the organism such that the gene is expressed in the organism. Methods for making transgenic plants and animals are known in the art.

In another embodiment, the markers are used to identify genes associated with the trait of interest. Once one or more QTLs have been identified that are significantly associated with the expression of the gene of interest, then each of these loci and linked markers may also be further characterized to determine the gene or genes involved with the expression of the gene of interest, for example, using map-based cloning methods as would be known to one of skill in the art. For example one or more known regulatory genes can be mapped to determine if the genetic location of these genes coincide with the QTLs controlling mRNA expression of the gene of interest. Confirmation that such a coinciding regulatory gene is effecting the expression of one or more genes of interest can be obtained using standard techniques in the art, for example, but not limited to, genetic transformation, gene complementation or gene knock-out techniques, or overexpression. The genetic linkage map can also be used to isolate the regulatory gene, including any novel regulatory genes, via map-based cloning approaches that are known within the art whereby the markers positioned at the QTL are used to walk to the gene of interest using contigs of large insert genomic clones. Positional cloning is one such a method that may be used to isolate one or more regulatory genes as described in Martin et al. (Martin et al., 1993, Science 262: 1432-1436; which is incorporated herein by reference).

“Positional gene cloning” uses the proximity of a genetic marker to physically define a cloned chromosomal fragment that is linked to a QTL identified using the statistical methods herein. Clones of linked nucleic acids have a variety of uses, including as genetic markers for identification of linked QTLs in subsequent marker assisted breeding protocols, and to improve desired properties in recombinant plants where expression of the cloned sequences in a transgenic plant affects an identified trait. Common linked sequences which are desirably cloned include open reading frames, e.g., encoding nucleic acids or proteins which provide a molecular basis for an observed QTL. If markers are proximal to the open reading frame, they may hybridize to a given DNA clone, thereby identifying a clone on which the open reading frame is located. If flanking markers are more distant, a fragment containing the open reading frame may be identified by constructing a contig of overlapping clones. However, other suitable methods may also be used as recognized by one of skill in the art. Again, confirmation that such a coinciding regulatory gene is effecting the expression of one or more genes of interest can be obtained via genetic transformation and complementation or via knock-out techniques described below.

Upon identification of one or more genes responsible for or contributing to a trait of interest, transgenic plants can be generated to achieve the desired trait. Plants exhibiting the trait of interest can be incorporated into plant lines through breeding or through common genetic engineering technologies. Breeding approaches and techniques are known in the art. See, for example, Welsh J. R., Fundamentals of Plant Genetics and Breeding, John Wiley & Sons, NY (1981); Crop Breeding, Wood D. R. (Ed.) American Society of Agronomy Madison, Wis. (1983); Mayo O., The Theory of Plant Breeding, Second Edition, Clarendon Press, Oxford (1987); Singh, D. P., Breeding for Resistance to Diseases and Insect Pests, Springer-Verlag, NY (1986); and Wricke and Weber, Quantitative Genetics and Selection Plant Breeding, Walter de Gruyter and Co., Berlin (1986). The relevant techniques include but are not limited to hybridization, inbreeding, backcross breeding, multi-line breeding, dihaploid inbreeding, variety blend, interspecific hybridization, aneuploid techniques, etc.

In some embodiments, it may be necessary to genetically modify plants to obtain a trait of interest using routine methods of plant engineering. In this example, one or more nucleic acid sequences associated with the trait of interest can be introduced into the plant. The plants can be homozygous or heterozygous for the nucleic acid sequence(s). Expression of this sequence (either transcription and/or translation) results in a plant exhibiting the trait of interest. Methods for plant transformation are well known in the art.

The following examples are offered by way of illustration and not by way of limitation.

### EXPERIMENTAL EXAMPLES

### Example 1

### Step By Step Processing of NPM Analysis Results For Picking Significant QTLs Using SAS

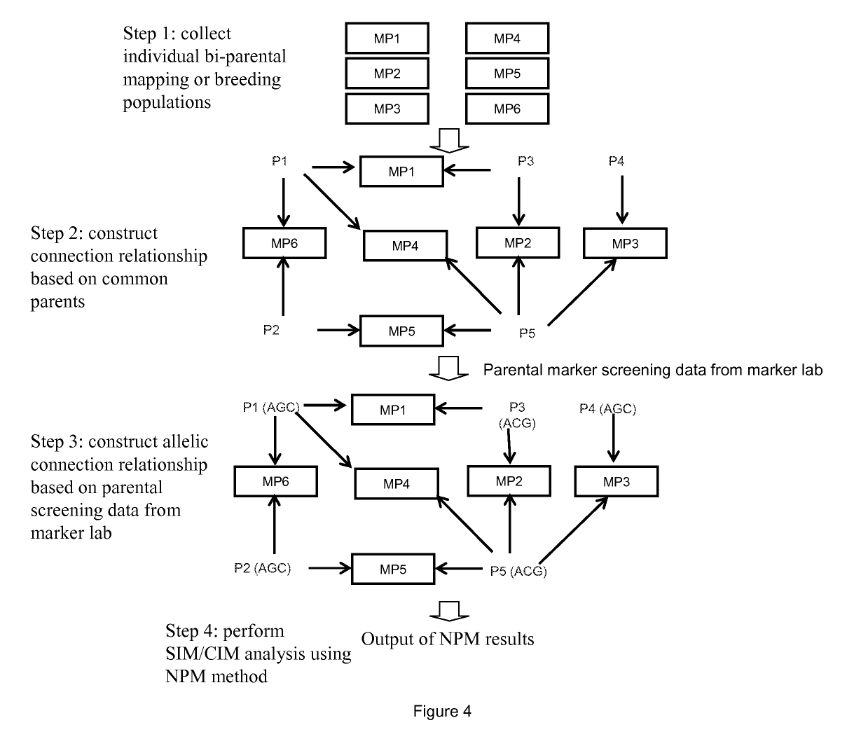

The steps of NPM analysis are outlined in FIG. 4. Individual bi-parental mapping or breeding populations are collected. A connection relationship is constructed based on common parents in the population. Allele information is collected at each of a series of marker loci for each member of the population. Allelic relationships are constructed based on this allele information, and NPM analysis is performed based on the relationship of the individuals at the allele level. The following steps are performed to assemble the data and run the NPM analysis:
1. As more than one allele per locus exists, multiple rows will accommodate the information pertaining to multiple alleles. Allele data is collected for each member at each marker locus. Hence as a first step, these input files were compressed into a smaller size table such that each row has all the information related to the corresponding hypothesis test (crosswise).
2. For each of the traits under study, only the rows with LOD values higher than the LOD threshold value (calculated from 1000 permutations) were retained for the purpose of locating the QTL peaks.
3. This data passes through a SAS code that scans all the chromosomes from top to bottom and identifies QTL peaks based on the sudden drop in the LOD score that follows a peak.
4. An interval of 2 cM from either side of the QTL peaks is also scanned for defining the approximate 95% confidence intervals (CI). The LOD and map position values from these intervals will be populated for all the QTLs detected in the earlier step.
5. Traits under study are assigned either a ‘+’ or ‘−’ sign based on whether a breeder generally selects for higher values or lower values in the segregating progeny.

The above criteria, along with the absolute allele effect values of the detected QTLs, are then used to develop a ranking order for both QTLs and their allele effects.
6. For each of the traits, all the QTLs detected across all chromosomes are ranked based on the sum value of the product of LOD value and the absolute maximum additive value observed from all alleles tested at QTL positions.
7. For allele ranking, if the trait under study is positive, then allele with highest effect is considered as the most favorable and otherwise, the allele with smallest effect is the most favorable one. At each of the QTL peaks, multiple allele effects are sorted either in descending (for positive traits) or in ascending order (for negative traits) based on the trait sign. The sorting order thus generated gets assigned as ranking number to the alleles tested at that QTL position.
   The output files for this process include:
1. A comma separated values format file with genome-wide LOD scans from the NPM analysis at 1 cM interval. The first few columns from this scans table consists of information used for the hypothesis testing such as the trait under study, number of member populations included from the network, genetic position on the chromosome, left and right locus names along with their haplotype states. It also has the information of NPM estimated parameters—namely LOD value, allele effect, percent trait variation explained, and names of member parents having the combination of flanking haplotype alleles involved in the hypothesis testing. These scan files are generally very lengthy tables, but can be easily read and managed in subsequent steps.
2. Results from 1000 NPM model analysis permutations performed for each of the selected traits involved in the study are provided in a MS Excel table or in a comma separated values format.
3. A tab delimited text file is created with information about linkage groups/chromosomes along with names of polymorphic loci and their consensus map positions. This file has the same genetic map information that was supplied earlier for the NPM analysis but in a different format.

### Example 2

### Step By Step Process of the Comparison With the Bi-Parental QTL Mapping vs NPM

A comparison effort was carried out to identify the differences between bi-parental versus connected mapping analyses. For this comparison, results from three different bi-parental CIM mapping models namely, 0%, 1% and 5% co-factor models, were compared against CPM and NPM connected analyses. The detailed description of the above mentioned CIM bi-parental mapping models can be found in the Win QTLCart documentation (which can be found on the internet at statgen.ncsu.edu/qtlcart/HTML/index.html). As a first step, all the bi-parental mapping analyses of the member populations were rerun using QTLCart software using a consensus map instead of their individual genetic maps.

The comparison was carried out in two different ways: 1) by comparing the whole genome scan visuals; and, 2) by comparing the estimates of QTLs detected with each of these methods.

### 1. Comparison of the Whole Genome Scan Visuals:

A Visual Basic macro was designed which takes the input of the LOD values observed across chromosomes (from mapping analyses) and displays them as heat graphs in MS Excel. Using this tool, the genome wide patterns of LOD values from different mapping models can be aligned side by side. So, the mapping results from CPM, NPM and bi-parental methods were fed into the macro to view the LOD score patterns along different chromosomes.

### 2. Comparison of Estimated QTL Parameters Across Different Mapping Methods:

A comparison of CPM, NPM and the corresponding individual bi-parental mapping analyses was also performed on the basis of the number of QTLs detected, mean observed LOD score, R-square values etc. For identifying the QTLs that agree with bi-parental results, the 95% QTL CIs from connected analysis were compared with 95% QTL CIs from the individual populations. This number was then subtracted from the total number of QTLs to get the number of QTLs uniquely identified in connected analysis. A weighted percentage of new QTLs detected were calculated by dividing the new connected analysis QTLs with the sum of total number of QTLs and new connected analysis QTLs.

### Example 3

### Experimental Examples For One Network But at Least Two Traits of Interest.

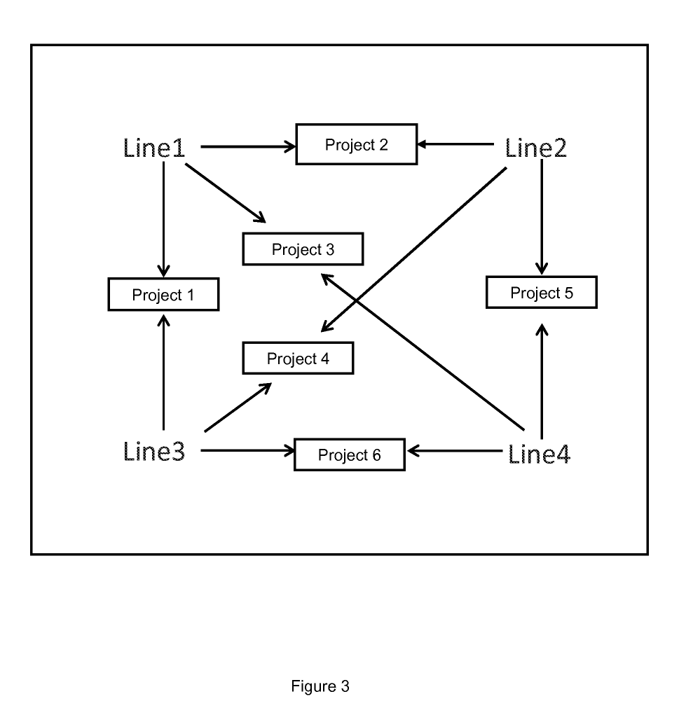

Analysis was done on a network consisting of six F₄ mapping populations derived from 4 parental lines and each consisting of 180 progeny. FIG. 3. Testcross hybrid data was collected for grain moisture and yield traits from five different field locations/environments. These traits were chosen based on their general heritability nature (yield—low heritable and grain moisture—high heritable). The data from each of these mapping populations was formatted in to the standard .mcd input file used for Win QTLCart and then was submitted for connected analysis. Two more input files (the consensus map and parental allele information) are also supplied for carrying out the connected mapping analysis.

The output files obtained from the connected analysis (both CPM and NPM) were processed through a SAS program (as described in Example 1) to list the QTLs. The output table contains a summary of the number of QTLs detected for the two traits of interest across 5 locations (Table 1). Row 3 represents the total number of QTLs detected in the analysis. Row 4 represents the total number of QTLs that were also detected in the biparental analysis. Row 5 represents the total number of new QTLs identified using CPM or NPM compared to biparental analysis. Row 6 represents the weighted percentage of new QTLs detected in CPM or NPM compared to biparental analysis.

TABLE 1

| | Model | | | |
|---|---|---|---|---|
| | CPM_CIM | NPM_CIM | CPM_CIM | NPM_CIM |
| Trait | Grain | Grain | Yield | Yield |
| | Moisture | Moisture | | |
| Total_Connected_Analysis_QTLs | 31 | 34 | 6 | 7 |
| QTLs_Agree | 27 | 26 | 5 | 4 |
| with_Biparent_results | | | | |
| New_Connected_Analysis_QTLs | 4 | 8 | 1 | 3 |
| WPer_NewConnected_Analysis_QTLs | 11.43 | 19.05 | 14.29 | 30 |

Table 2 presents the results of this analysis in terms of LOD score and absolute allelic effects. Row 3 represents the average LOD score in the connected analysis. Row 4 represents the average LOD score in the biparental analysis. Rows 5 and 6 represent the absolute allele effect values for connected analysis (row 5) and biparental analysis (row 6). Rows 7 and 8 represent the average percent of variation explained by the QTLs for connected analysis (row 7) and biparental analysis (row 8).

TABLE 2

| | Model | | | |
|---|---|---|---|---|
| | CPM_CIM | NPM_CIM | | |
| | Grain | Grain | CPM_CIM | NPM_CIM |
| Trait | Moisture | Moisture | Yield | Yield |
| Connected_Analysis_Avg_LOD | 7.63 | 5.13 | 5.25 | 4.18 |
| Biparental_Analysis_Avg_LOD | 5.24 | 5.86 | 3.52 | 3.58 |
| Connected_Analysis_Avg_Abs_Add | 0.22 | 0.2 | 1.76 | 1.62 |
| Biparental_Analysis_Avg_Abs_Add | 0.54 | 0.54 | 4.06 | 3.65 |
| Connected_Analysis_Avg_R2 | 3.19 | 13.91 | 2.22 | 11.43 |
| Biparental_Analysis_Avg_R2 | 13.84 | 13.65 | 10.55 | 9.24 |

The conclusions from the whole genome scan visual comparison are
1. Both the CPM and NPM models gave consistent LOD score patterns across the genome, despite the fact that shared allele information is not modeled in the CPM model. Differences between CPM and NPM results are expected in the number of alleles involved for the hypothesis testing and in the estimation of the allele effect.
2. There is good visual correlation in QTL detection between bi-parental mapping and connected mapping analyses (both CPM and NPM).
3. Whenever a QTL was detected in at least one of the members of the network, a corresponding QTL also appeared in the connected analysis.
4. At connected analysis QTL positions, the observed LOD values are proportional to the QTL positions detected in member populations.
5. There are some QTLs detected in connected analysis that were not observed in any of the bi-parental analyses.
   The conclusions from the comparison of estimated QTL parameters are as follows:
1. In general, for both high and low heritable traits, the number of QTLs detected increased from the CPM to the NPM model (Table 1, columns 3 and 5).
2. The mean of LOD values observed at the QTL positions were higher in the case of CPM analysis compared to their corresponding bi-parental results. However, the mean of LOD values observed at the QTL positions of NPM results were comparable to those observed from the bi-parental results (Table 2, rows 3 and 4).
3. The absolute allele effect values in the case of CPM and NPM analyses (estimated using random model) were lower compared to the absolute allele effects observed in individual bi-parental mapping analyses (estimated using fixed model) (Table 2, rows 5 and 6). This is an expected trend as allele effects estimated using marker genotypes as fixed model tend to be biased.
4. The average percent of variation explained by the QTLs from the CPM model were less than those obtained from bi-parental mapping analyses (Table 2, rows 7 and 8, columns 2 and 4). However, the NPM model gave the best QTL average percent r-square estimates (Table 2, rows 7 and 8, columns 3 and 5). This trend is also expected due to increased sample size and inclusion of shared alleles in the NPM analysis.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A method for evaluating an association between a marker and a trait of interest in a connected population of organisms comprising:
a) determining the haplotype for at least one polymorphic marker for each member of said population;
b) determining the phenotypic value for said trait of interest for each member of said population;
c) grouping members of said population according to shared haplotypes for said at least one polymorphic marker;
d) determining whether said marker is associated with said trait of interest in the network selected in step (c).

2. The method of claim 1, wherein step (d) comprises an interval-based association model.

3. The method of claim 1, wherein step (d) comprises an association model comprising a means for estimating and ranking the effects on the trait of interest of individual haplotypes of said marker across said connected population.

4. The method of claim 2, wherein said effects of individual haplotypes are treated in said association model as random effects.

5. The method of claim 1, wherein step (d) comprises an association model comprising a means for accounting for the effect on the trait of interest of different genetic backgrounds represented in said population.

6. The method of claim 5, wherein said effect is a fixed effect.

7. The method of claim 3, wherein said model consists of:
yᵢⱼ=μ+zᵢⱼa^{q}+gᵢ+eᵢⱼ,
where yᵢⱼ is the phenotypic value of the individual j in the population i;
wherein μ is the overall mean;
wherein zᵢⱼ is the indicator variable showing whether the allele q comes from the population i;
wherein a^{q} is the effect of the allele q of a QTL;
wherein gᵢ is the effect of the polygenetic background from the population i;
wherein eᵢⱼ is the residual term;
wherein the effect of the allele q is a random effect; and
wherein the effect of the allele q is calculated using best linear unbiased prediction (BLUP).

8. The method of claim 3, wherein said model consists of:
yᵢⱼ=μ+zᵢⱼa^{q}+Σ(k=1, c)xᵢⱼₖbₖ+gᵢ+eᵢⱼ,
where yᵢⱼ is the phenotypic value of the individual j in the population i;
wherein μ is the overall mean;
wherein zᵢⱼ is the indicator variable showing whether the allele q comes from the population i;
wherein a^{q} is the effect of the allele q of a QTL;
where xᵢⱼₖ is the genotype of the cofactor marker k of the line j in the population i;
wherein bₖ is the effect of the marker k;
wherein gᵢ is the effect of the polygenetic background from the population i;
wherein eᵢⱼ is the residual term;
wherein the effect of the allele q is a random effect; and
wherein the effect of the allele q is calculated using best linear unbiased prediction (BLUP).

9. The method of claim 8, wherein the cofactor markers are selected based on a defined significance level.

10. The method of claim 9, wherein said significance level is less than or equal to 0.1.

11. The method of claim 8, wherein cofactors are selected using a model comprising:
yᵢⱼ=μ+Σ(k=1, c)xᵢⱼₖbₖ+gᵢ+eᵢⱼ
wherein yᵢⱼ is the phenotypic value of the individual j in the subpopulation i;
wherein μ is the overall mean;
where xᵢⱼₖ is the genotype of the cofactor marker k of the line j in the population i;
wherein bₖ is the effect of the marker k;
wherein gᵢ is the effect of the polygenetic background from the population i; and
wherein eᵢⱼ is the residual error.

12. The method of claim 1, wherein said connected population is a diallel, a partial diallel, or a combination of a diallel and a partial diallel cross of a plurality of inbred lines.

13. The method of claim 1, wherein said population of organisms is a plant population.

14. A method for breeding a population of organisms exhibiting a trait of interest comprising:
a) determining the haplotype for a plurality of polymorphic markers for each member of a population of said organisms;
b) determining the phenotypic value for said trait of interest for each member of said population;
c) grouping members of said population according to shared haplotypes for at least a first polymorphic marker;
d) determining whether said at least a first polymorphic marker is associated with said trait of interest in the network selected in step (c);
e) repeating steps (c) and (d) for one or more polymorphic markers until at least one marker is determined to be associated with said trait of interest;
f) identifying an organism comprising the marker that is associated with said trait of interest;
g) crossing the organism identified in step (f) with a compatible organism of interest;
h) selecting progeny from said cross by selecting for the presence of said marker associated with said trait of interest; and
i) breeding the progeny selected in step (h) to obtain said population of organisms exhibiting said trait of interest.

15. The method of claim 14, wherein said marker that is associated with said trait of interest comprises a favorable allele for said trait of interest.

16. The method of claim 14, wherein step (d) comprises an interval-based association model.

17. The method of claim 14, wherein step (d) comprises an association model comprising a means for estimating and ranking the effects on the trait of interest of individual haplotypes of said marker across said connected population.

18. The method of claim 17, wherein said effects of individual alleles are treated in said association model as random effects.

19. The method of claim 14, wherein step (d) comprises an association model comprising a means for accounting for the effect on the trait of interest of different genetic backgrounds represented in said population.

20. The method of claim 19, wherein said effect is a fixed effect.

21. The method of claim 17, wherein said model consists of:
yᵢⱼ=μ+zᵢⱼa^{q}+gᵢ+eᵢⱼ,
where yᵢⱼ is the phenotypic value of the individual j in the population i;
wherein μ is the overall mean;
wherein zᵢⱼ is the indicator variable showing that if the allele q comes from the population i;
wherein a^{q} is the effect of the allele q of a QTL;
wherein gᵢ is the effect of the polygenetic background from the population i;
wherein eᵢⱼ is the residual term;
wherein the effect of the allele q is a random effect; and
wherein the effect of the allele q is calculated using best linear unbiased prediction (BLUP).

22. The method of claim 17, wherein said model consists of:
yᵢⱼ=μ+zᵢⱼa^{q}+Σ(k=1, c)xᵢⱼₖbₖ+gᵢ+eᵢⱼ,
where yᵢⱼ is the phenotypic value of the individual j in the population i;
wherein μ is the overall mean;
wherein zᵢⱼ is the indicator variable showing whether the allele q comes from the population i;
wherein a^{q} is the effect of the allele q of a QTL;
where xᵢⱼₖ is the genotype of the cofactor marker k of the line j in the population i;
wherein bₖ is the effect of the marker k;
wherein gᵢ is the effect of the polygenetic background from the population i;
wherein eᵢⱼ is the residual term;
wherein the effect of the allele q is a random effect; and
wherein the effect of the allele q is calculated using best linear unbiased prediction (BLUP).

23. The method of claim 22, wherein the cofactor markers are selected based on a defined significance level.

24. The method of claim 23, wherein said significance level is less than or equal to 0.1.

25. The method of claim 22, wherein cofactors are selected using a model comprising:
yᵢⱼ=μ+Σ(k=1, c)xᵢⱼₖbₖ+gᵢ+eᵢⱼ
wherein yᵢⱼ is the phenotypic value of the individual j in the subpopulation i;
wherein μ is the overall mean;
where xᵢⱼₖ is the genotype of the cofactor marker k of the line j in the population i;
wherein bₖ is the effect of the marker k;
wherein gᵢ is the effect of the polygenetic background from the population i; and
wherein eᵢⱼ is the residual error.

26. The method of claim 14, wherein said connected population is a diallel, a partial diallel, or a combination of a diallel and a partial diallel cross of a plurality of inbred lines.

27. The method of claim 14, wherein said population of organisms is a plant population.

28. The method of claim 14, wherein said polymorphic markers are candidate genes.

29. The method of claim 28, further comprising introducing into an organism an expression construct comprising said marker associated with said trait of interest, wherein said nucleic acid is operably linked to a promoter functional in the organism into which said construct is introduced, and wherein said organism thereby exhibits the trait of interest.

30. The method of claim 29, wherein said organism is a plant.
